(19) United States
(12) Patent Application Publication
(10) Pub. No.: US 2010/0062521 A1
(43) Pub. Date: Mar. 11, 2010

(54) PACKAGING DEVICE FOR BIOLOGICAL ANALYSIS

(75) Inventors: Sylvie Veriac, Montpellier (FR); Valérie Paya-Souquet, St Gely du Fesc (FR); François Dupoteau, Paris (FR)

Correspondence Address: BROWDY AND NEIMARK, P.L.L.C.;624 NINTH STREET, NW, SUITE 300, WASHINGTON, DC, 20001-5303, US

(73) Assignee: Horiba ABX SAS, Montpellier Cedex 4 (FR)

(21) Appl. No.: 12/301,231

(22) PCT Filed: May 15, 2007
(86) PCT No.: PCT/FR2007/000821

§ 371 Date: Jul. 6, 2009

(30) Foreign Application Priority Data
May 16, 2006 (FR) 0604336

(51) Int. Cl. C12M 1/34

(52) U.S. Cl. 435/288.1

(57) ABSTRACT

A subject of the invention is a packaging device for automated biological analyses, characterized in that it comprises the sample(s) to be analyzed, reagent(s), and the information necessary to carry out one (or more) analyse(s) of a sample of biological liquid and a location intended to receive the waste products from the analysis and optionally to treat them.

The present invention relates to the field of analysis of biological liquids and more particularly the field of automatic machines for analyses of biological liquids. It relates very particularly to a device (or support) intended to be introduced into an automatic analysis machine, said device bringing together not only the sample to be analyzed and the reagents necessary to analyze said sample and/or to neutralize the waste products but also the information necessary for said analysis to be carried out by the automatic machine, and at least one container intended to receive and optionally neutralize the waste products at the end of the analysis.

Devices intended to support samples before being analyzed by an automatic machine are known in the state of the art.

The European Patent Application EP0567093 of TORAY INDUSTRIES describes a rack system containing several cartridges each containing three reaction tubes, a dilution tube and a tube for the sample. This device, intended for an automatic analysis machine, requires an additional unit called “reagent dispensing unit” which will dispense the reagents into a container, and distributes them in said rack cartridges using motorized syringes.

Moreover, this device does not comprise an integrated information system suitable for the analysis to be carried out on the sample and does not provide a container intended to receive the waste products at the end of the analysis.

The International Application WO8/606488, of HICHEM DIAGNOSTICS, describes a rectangular analysis kit comprising several recesses in which closed containers are arranged, made of plastic material, containing different reagents or antibodies according to the required analysis. The base of the cavities has a pointed protrusion intended to rupture the lower surface of said container when a pressure is applied by a technician on the upper surface of said container, thus releasing the reagent.

This kit only allows a simple type of analysis, and above all requires the intervention of a person who releases the reagents according to an established order according to the protocol of the desired analysis.

Moreover, this kit does not provide integrated means for quality control, is not suitable for an automatic analysis machine and does not provide a container intended to receive the waste products at the end of the analysis. No method for reading the results is given.

The Patent Application DE4313807A of Olympus Optical describes a compact system which can contain reagents or antibodies comprising a barcode system containing specific information about period of validity, of use, etc. The blood-reagent reaction is not carried out in the containers of the system. This system does not comprise a container intended to receive the waste products at the end of the analysis.

The U.S. Pat. No. 5,788,928 of CHIRON DIAGNOSTIC CORPORATION describes a pack with a specific shape which facilitates the mixing of liquids, intended to receive doses of reagents. A device for agitation by rocking allows the reagents to be mixed automatically.

The reagents are not included directly in the pack and its shape is provided for integration into a specific analysis device. Finally, this device does not comprise an information system suitable for the analysis to be carried out on the sample or a container intended to receive the waste products at the end of the analysis.

The U.S. Pat. No. 5,578,272 of HOFFMAN LA ROCHE describes the characteristics of a kit of reagents integrated into a cartridge which can be used in an automatic biochemical analysis machine. The sample to be analyzed is not included in the cartridge. The reagents, integrated into the cartridge, are pierced by a needle which takes the liquid and transfers it into a reaction vessel, itself not included in the cartridge. The kit is also equipped with a barcode which allows the data relating to the reagents to be read. The mixing of the reaction is carried out separately using an agitation system. The reaction vessel is not integrated in the pack. The barcode does not contain information about the type of analysis to be carried out or about the method to be used. This kit does not comprise a container intended to receive the waste products at the end of the analysis.

Although devices for the automatic analysis of liquid samples exist, it is understood that it there remains a requirement for a device which brings together not only the sample to be analyzed and the reagents necessary to analyze said sample, but also a container which allows recovery of the waste products and, optionally, their neutralization, but also the information necessary to carry out said analysis by the automatic machine, particularly within the framework of biological analyses.

A subject of the invention is a packaging device for biological analyses, characterized in that it comprises at least one sample to be analyzed, at least one reagent, at least one container allowing recovery of the waste products originating from the analysis, and at least one information system containing the information necessary to carry out at least one analysis of said sample to be analyzed.

The device according to the invention is particularly well-suited to the analysis of sample(s) of biological liquids, particularly blood, very particularly of whole blood.

The device according to the invention has the advantage that it can be prepared in advance. Thus it contains as available reagent (or reagents) necessary for the analysis (or analyses) which must be carried out, optionally already diluted and ready for use in the quantity required and sufficient to carry out the envisaged analysis (or analyses), as well as the set of information required by the automatic machine into which it is introduced to carry out the analysis (or analyses). Moreover, it allows recovery of the waste products of the analysis. In this respect, the device according to the invention can also comprise at least one means for neutralizing the waste products.

Thus prepared, the device can be stored under conditions compatible with the preservation of the elements it contains until the sample to be analyzed is introduced into it.

In a particular form of use of the device according to the invention, the quantity of sample to be analyzed required for the analysis the parameters of which are stored in the information means can be introduced into the device by the operator prior to the introduction of the device into the automatic machine.

The device according to the invention therefore allows the greatest possible reduction in intervention by an operator who must do nothing more than introduce the sample to be analyzed into the device, place said device in the automatic machine and start it, whereupon the automatic machine interprets the information contained in the information means and carries out the analysis.

In another form of use of the device according to the invention, the sample to be analyzed can be introduced into the device and the quantity of said sample necessary for analysis the parameters of which are stored in the information means can be taken automatically by the automatic machine. It is understood that in this version, the information means also comprises information about the quantity of sample to be taken by the automatic machine.

The subject of the invention is particularly useful in an automatic machine of small size. It allows specific analyses to be carried out at low cost. Moreover, the presence of the information system directly on the support, as well as all the reagents in the containers, allows the invention to be used in a completely autonomous and reliable manner. In the case of a use in an automatic machine, the operator does not need to intervene to enter or select data relating to the measuring method or the reagents, which constitutes a saving in time and a reduction in handling errors.

The subject of the invention can be used one or more times. It can be disposed of after analysis or reused once cleaned and sterilized. This type of device is particularly suitable for specific analyses or for equipment which is not heavily used (concept of the <<Doctor Test>>).

The device according to the invention is particularly suitable for immunotyping analyses using antibodies.

More precisely, the device according to the invention comprises
at least one location intended to receive a reagent,
at least one location intended to receive a biological sample for analysis;
at least one location intended to receive the waste products originating from the analysis;
and at least one support for the information required to carry out said analysis by the automatic machine.

According to the invention at least one of the reagents can be an agent for neutralizing the waste products of the analysis.

According to a particular embodiment of the invention, the neutralizing agent can be introduced directly into the location intended to receive the waste products.

In a particular embodiment, the device according to the invention can also comprise at least one location intended to receive a reference.

In yet another particular embodiment, the device according to the invention can also comprise at least one location in which the reactions required for the analysis are carried out.
In the present text the concepts below are defined as follows:
support: the housing holding the set of containers together. This housing also being the support for the information system.
container: a tube or a cavity produced directly in the support, being able to contain the reagent(s), the reference(s) and the sample(s), the quality control(s) and the waste products of the analysis. The set of containers can be integrated in the support;
reagents: the set of products required for the sought reaction and/or for neutralizing the waste products, for example the chemical compounds, antibodies, dyes or also enzymes;
reference: the reagent or reagents allowing the calibration of the device and/or the positive or negative control of the analysis. This is a reference measurement and/or reaction which allows an internal quality control;
information system: any device which allows the reading and/or the writing of information relating to the sought analysis, particularly by the automatic analysis machine:
whole blood analyzer: the analyzers carrying out analyses on a tube of blood comprising all the elements of blood as opposed to equipment working on plasma or serum (equipment called “for biochemistry”). A cell counter can be mentioned as an example of a whole blood analyzer;
incubator: a space allowing a solution contained in a container to be stored and optionally agitated for the period necessary for chemical or biochemical reaction. The temperature, luminosity, pressure and agitation being able to be controlled inside the incubator;
pre-analytic functions: the actions which prepare the sample before its passage into the whole blood analyzer. The actions on the sample of whole blood being able to be the aeration of the contents, agitation, reading the information relating to the analysis, quality control management, looking for clots in the sample and moving the samples, controlling the level of blood in the contents, preparing a chemical reaction required for incubation, adjusting the sample to a certain temperature;
diagnostic profile: linking several hematological, biochemical and/or immunological parameters allowing diagnosis to be directed;
agent neutralizing the waste products: any chemical or biological or organic molecule, or any composition containing one of these molecules in solid or liquid form, allowing at least one of the reaction compositions to be deactivated. For example an agent neutralizing microorganisms, or also a disinfectant or a detoxifier allowing the environment to be respected.

According to the invention, the information support can be any means capable of storing information relating to the analysis to be carried out. It can be placed on the support and can be read by a suitable reader, optionally integrated into the analysis device in which the device according to the invention will be integrated. Thus, said information support can be a barcode, an electronic chip such as for example an RFID chip, a magnetic band or label or also a combination of at least two of these elements.

Said information support can comprise information relating to the measurement method and/or the use of the reagents and/or the processing of the sample to be analyzed. It can also comprise any information relating to the preservation of the products that it contains such as in particular an expiry date. For example, the information system can comprise the information necessary for starting and/or maintaining agitation and/or for the incubation necessary for the reaction to progress correctly, and/or information about the method of use and/or the characteristics of the reagent(s) contained in the device, and/or can also allow calibration to take place and/or quality control of the different types of analysis to be carried out and/or allow the history of the information to be established. It can also comprise any information necessary for managing and/or neutralizing the waste products.

In a particular embodiment, the device according to the invention can also comprise at least one or more mean(s), optionally mechanical, which make it possible to facilitate handling and/or agitation and/or thermal regulation of the substances contained in the device.

By means making it possible to facilitate manipulation is meant any means which allows the device to be introduced into or withdrawn from the analyzer, which include a sliding rail or a side flap allowing access to the device.

By means allowing agitation is meant a mechanical system formed from a rigid platform and a mobile shaft on which the device is positioned and allowing a rotation of this device on this shaft.

By means allowing thermal regulation is meant any means which allows the sample to be analyzed and/or the reagent or reagents to be taken to a temperature compatible with the analysis to be carried out. This means can therefore be a heating or refrigerating means. In this respect, there can be mentioned a Peltier apparatus for this purpose.

According to the invention, the support can be rigid or flexible. It can be made of any material having a good resistance to temperature, to shocks and/or to chemical reagents. It can preferably be made of plastic material.

Among these so-called engineered plastic materials, there can be mentioned the main fluorocarbon resins including polytetrafluoroethylene (PTFE), fluorinated ethylene propylene (FEP), perfluoroalkoxy (PFA), polyfluorovinylidene (PVDF), a modified copolymer of ethylene and tetrafluoroethylene (ETFE) or also ethylene/chlorotrifluoroethylene (ECTFE). Certain high-density polyethylene-type thermoplastics (PEHD) can also be used.

According to the invention, the device can have all conceivable shapes provided they are compatible with the automatic machine intended to receive them. A person skilled in the art knows how to adapt the shape of the device according to said automatic machine.

According to the invention, the locations in the device intended to receive the reagent or sample to be analyzed or the waste products or optionally the location where the reaction takes place, simultaneously or not, can be a cavity of any shape which can receive the reagent and/or sample and/or the waste products and/or the reaction mixture directly or a container, for example a tube, in which the reagent and/or sample and/or the waste products and/or the reaction mixture are located.

It is understood that according to requirements, one or other of said locations, or both simultaneously, can receive the reagent and/or sample directly or indirectly. There may be mentioned by way of example a device in which the reagent(s) are introduced directly into the location(s) intended to receive them and the sample is introduced into a container, for example a tube, itself introduced into the location of the device intended to receive it.

According to the invention, the location intended to receive the sample of biological liquid to be analyzed is fashioned so as to receive a container in which said sample is located.

In a particular embodiment in which the reagent and/or sample is (are) directly introduced into the location, which can moreover receive any means allowing it to be closed. There may be mentioned for example a stopper which adapts perfectly to the location, optionally screwing into the location, or also a membrane seal made of any suitable material, sealed over the device in order to completely block the location containing the reagent.

It is understood that the means allowing closure of the location in which the reagent is located can either be removed before introduction of the device into the automatic machine or be provided in a material that can be pierced for example by a needle controlled directly by the automatic machine.

The device according to the invention can be used in any biological analysis device including one using a manual mode. The device can advantageously be used in an automatic or semi-automatic apparatus. Among the apparatuses in which the device according to the invention can be used there may be mentioned an automatic hematology machine.

The device according to the invention can be envisaged in at least two versions:

A first version in which the support comprises a series of locations all containing the same reagent allowing a series of identical tests and one location containing a reference. In this version, several different samples can therefore undergo the same analysis.

A second version in which the support comprises a series of locations each containing different reagents each allowing a single test and one location containing a reference. In this version, the same sample can therefore undergo several treatments.

A subject of the invention is also a biological analysis process characterized in that it implements a device as described previously.

A subject of the invention is also an automatic biological analysis machine, particularly an automatic machine for the analysis of biological liquid, characterized in that it implements a device as described previously.

The figures show the support in a certain configuration which can vary according to the implementation. In particular, the shape of the support is indicative only; a circular support in which the containers are positioned in a circle is conceivable. Moreover, the shape, the number and the arrangement of the different containers can vary.

The subjects, characteristics and advantages of the invention follow from the description below, given by way of illustrative, non-limitative example, with reference to the attached drawings in which:

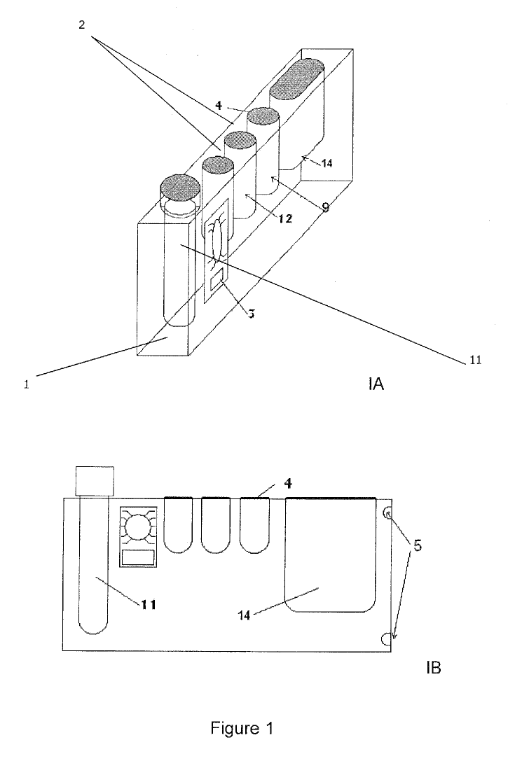

FIG. 1 represents a device according to the invention, shown in perspective (1A) or side (1B) view,

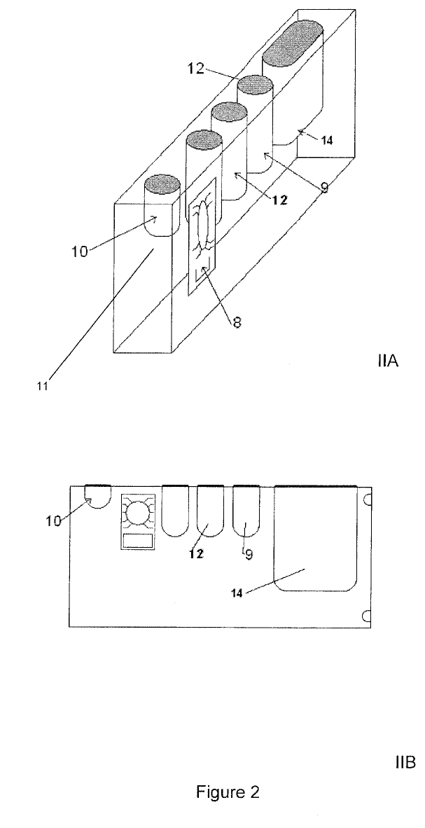

FIG. 2 shows a device according to the invention in perspective (2A) or side (2B) view.

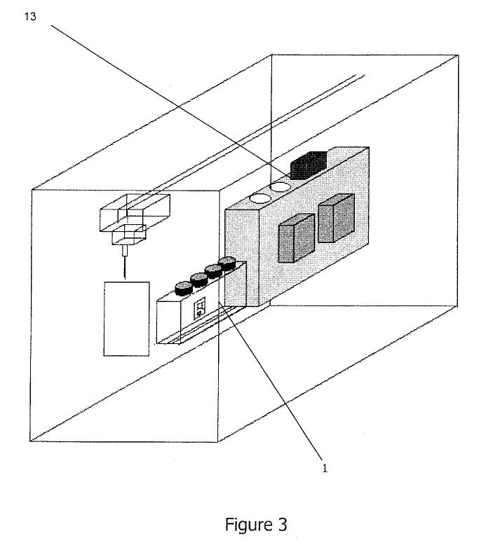

FIG. 3 shows diagrammatically an analyzer with a integrated device according to the invention.

FIG. 1) corresponds to a device according to the invention, presented in perspective (1A) or lateral (1B) view, laid out with integrated containers 2 and a location 11 configured to accommodate a conventional tube intended for the sample.

The device 1 is made of rigid plastic, the locations 2 and 4 are moulded directly inside the support and are an integral part of the latter. Two types of locations are shown here, a location 4 intended to receive the reference and locations 2 containing the reagents intended to be analyzed. The information system 3 (in this case an RFID chip), containing the information regarding the reagents and the method of use is arranged along the device in order to be able to be read by a suitable reader arranged in the analyzer. The locations which are closed in a hermetic manner are pierced with or without impact so as to allow access to the reagent.

The doses of reagent(s) necessary for the analysis are then automatically transferred to the location containing the sample where the reaction takes place. Such a device also allows the analysis to be carried out by automatic transfer of a predetermined dose of the sample to be analyzed into each of the locations which then contains the required dose of reagent.

In this example the device is also equipped with a container 14 intended to receive, after use, the biological and chemical liquids used during analysis of the sample, this container being able to be equipped with a solid or liquid reagent which will allow the waste products of the analysis to be neutralized.

In this example, the device is accommodated in dedicated a compartment in the apparatus and it is positioned by means of several notches 5.

FIG. 2 shows a device according to the invention 1 in perspective (2A) or side (2B) view, laid out with the locations integrated in the support. The device 1 is made of rigid plastic in this case, the locations 9, 10 and 12 are moulded directly inside the support and form an integral part of the latter. Three types of locations are shown here, a location 9 intended to receive the reference, a location 10 intended to receive the sample to be analyzed and locations 12 containing the reagents intended for analysis. The information system 8 (in this case, an RFID chip) containing the information regarding the reagents and the method of use, is arranged along the support in order to be able to be read by a suitable reader arranged in the analyzer. The locations which are closed in a hermetic manner are pierced with or without impact so as to allow access to the reagent.

The methods of use of this variant of the device according to the invention are identical to those described for the variant of FIG. 1.

FIG. 3) shows diagrammatically an analyzer with a device integrated according to the invention.

Device 1 in automatic mode is in this case integrated in a corpuscle counter for whole blood equipped with a location 13 dedicated to this purpose. The device can be introduced into the corpuscle counter via the front of the latter but configurations can allow introduction by the side or by the rear. The device is positioned lengthways in the analysis block.

The examples below of utilization of the device according to the invention illustrate the invention without limiting it.

### EXAMPLE 1

A blood sample is analyzed on an automatic device according to the multiparameter methods described in French patent no. 01/02489. Following analysis of the leukocytes in a sample of whole blood, the presence of elements is observed in an area which does not allow them to be separated and counted precisely because of the proximity of adjacent sub-populations: monocytes and lymphocytes. This observation can generate an alarm on the analyzer or not, nonetheless an additional; control analysis proves necessary to separate and optionally identify the elements of this area.

In such a case, the control would consist of carrying out a smear and performing a cell count of the leukocyte differential count by microscope on a maximum of a few hundred cells. A purely estimated and statistical relationship is made between the obtained result and the result of the automatic analysis.

Although it is actually possible to obtain approximately the same result in flow cytometry, once again the direct relationship with the first analysis obtained routinely does not exist and reduces the usefulness and precision of the operation.

The invention allows an additional control to be brought to bear and therefore additional information to a first analysis. It consists of demonstrating by specific marking one or more sub-population(s) superimposed on the standard analysis. To carry out the differentiation in our example, the anti-CD45 antibody is used simultaneously with the routine analysis in order to positively mark the normal elements. This marker is expressed specifically on the leukocytic elements such as lymphocytes, eosinophilic leukocytes, monocytes, basophilic leukocytes and neutrophilic leukocytes (decreasing order of expression).

In this case, the device used is a monoreagent device (first version) containing the anti-CD45 antibody, reagents, optionally washing solutions, a reference and a location for receiving the waste products.

In a pre-analysis phase, it is possible to monitor the device using the reference contained in the device, this reference allows the quality of the optical measuring components to be verified before the sample is passed through. Given the duration of incubation of the sample, this calibration can be carried out in concurrent operation time.

This analysis is carried out in the following manner:
Place the device according to the invention into the apparatus
Read the information contained in the information system (in this case a chip) attached to the support
Enter the data relating to the analysis method and the properties of the reagent
Take a 50 μl sample of whole blood to be analyzed
Mix with 50 μl of anti-CD45 monoclonal antibody solution conjugated to a fluorescent dye in one of the containers of the device
Clean the sampling needle
Agitate the device in order to facilitate a better reaction
Incubate the solution in the device at ambient temperature, maintained by the temperature maintenance system around the device
Put a sample of the reference into the device
Calibrate the measuring cell during the incubation
Clean the sampling needle
Take a sample of the blood/antibody solution using the sampling analyzer needle.
Inject a nucleic acid dye reagent for routine analysis and leukocyte identification
Transfer the thus-obtained solution to the optical bench for analysis
Inject the solution into the optical cell with circulation in order to measure the following parameters
   Volume of elements by impedance (RES) allowing the cell count
   Measurement of light diffracted along the axis (FSC)
   Measurement of light diffused orthogonally (SSC)
   Measurement of orthogonal fluorescence of the nucleic acid dye (FL1)
   Measurement of the orthogonal fluorescence of the dye conjugated to the anti-CD45 antibody (FL2).
Acquire and process the data. Produce the results and imaging.
Return the solution containing the sample of blood and reagents used during analysis to the needle
Deposit them in the container intended for recovery and neutralization of the biological and chemical liquids originating from the analysis

The first four parameters are parameters used routinely and the last parameter (FL2) is reserved for cell identification verification functions as explained previously.

### EXAMPLE 2

In this version, the device according to the invention which is used contains several reagents plus a reference (second version).

It has been shown that the expression of the CD64 surface antigen on the population of neutrophilic leukocyte cells is a biological response characteristic of inflammatory phenomena.

Moreover, the CD163 surface antigen is highly expressed at the surface of macrophages. It is itself also characteristic of inflammatory phenomena.

The reagents used are 2 monoclonal antibodies (an anti-CD64 and an anti-CD163) which allow the inflammatory characteristics of the surface-antigenic expression of leukocyte cells to be demonstrated.

The reference is a suspension of fluorescent beads used to calibrate the instrument and to standardize the reactive method.

The analysis is carried out as follows:
Place the device into the apparatus
Read the information contained on the chip attached to the support of the device
Enter the data relating to the analysis method
Take a 50 μl sample of whole blood to be analyzed
Mix with 50 μl of anti-CD64 and anti-CD163 monoclonal antibody solution each conjugated to a different fluorescent dye in a container
Clean the sampling needle
Transfer a sample of the calibrating beads solution into the device
Agitate the device in order to facilitate a better reaction
Incubate the solution in the device at ambient temperature, maintained by the temperature maintenance system around the device
Calibrate the measuring cell during the incubation
Inject the erythrolytic solution (lysis of red corpuscles)
Take a sample of the blood/antibody solution using the sampling needle.
Transfer the thus-obtained solution to the optical bench for analysis
Inject the solution into the optical cell with circulation of the optical bench in order to measure the following parameters:
   Volume of elements by impedance (RES) and count
   Measurement of light diffracted along the axis (FSC)
   Measurement of light diffused orthogonally (SSC)
   Measurement of orthogonal fluorescence of the dyes conjugated to the anti-CD64 and anti-CD163 antibodies (FL1 vs FL2).
Acquire and process the data. Produce the results and imaging.
Return the solution containing the sample of blood and reagents used during analysis to the needle
Deposit them in the container intended for recovery and neutralization of the biological and chemical liquids originating from the analysis.

1. A packaging device for biological analyses (1), comprising at least one sample to be analyzed (10, 11), at least one reagent, at least one container (14) allowing recovery of the waste products from the analysis, and at least one information system (3, 8) containing the information necessary to carry out at least one analysis of said sample to be analyzed, characterized in that at least one of the reagents is an agent neutralizing the waste products from the analysis.

2. Device according to claim 1, further comprising:
at least one location intended to receive a reagent,
at least one location intended to receive a biological sample for analysis;
at least one location intended to receive the waste products originating from the analysis;
and a support for the information required to carry out said analysis by an automatic machine.

3. Device according to claim 1, wherein the biological sample(s) is/are a sample of biological liquids.

4. Device according to claim 1, wherein the neutralizing agent is introduced directly into the location intended to receive the waste products.

5. Device according to claim 1, further comprising at least one location intended to receive a reference.

6. Device according to claim 1, further comprising at least one location in which the reactions necessary for analysis are carried out.

7. Device according to claim 1, wherein the location intended to receive the reagent and/or sample to be analyzed and/or the waste products originating from the analysis and/or optionally the location where the reaction is carried out, is a cavity which can receive either directly the reagent and/or sample and/or waste products and/or reaction mixture or a container in which the reagent and/or sample and/or waste products and/or reaction mixture are located.

8. Device according to claim 7, wherein the information support makes it possible to store the information relating to the method of measurement and/or use of the reagents and/or processing of the sample to be analyzed and/or information relating to the preservation of the products that it contains.

9. Device according to claim 1, wherein the information contained in the information support is information necessary for starting and/or maintaining agitation and/or for the incubation necessary for the reaction to progress correctly, and/or information about the method of use and/or the characteristics of the reagent(s) contained in the device, and/or information which make it possible to manage calibration and/or quality control of the various types of analysis to be carried out and/or allows the history of the information to be established.

10. Device according to claim 1, wherein the information support is an electronic chip, a barcode, a magnetic label or a combination of at least two of these elements.

11. Device according to claim 1, further comprising a system, optionally mechanical, allowing the handling and/or agitation and/or thermal regulation of the substances contained in the device to be facilitated.

12. Device according to claim 1, wherein said device is made of a material having a good resistance to shocks and/or to chemical reagents, preferably made of plastic material.

13. Device according to claim 12, wherein said device is made of fluorocarbon resin.

14. Device according to claim 13, wherein the fluorocarbon resin is chosen from polytetrafluoroethylene (PTFE), fluorinated ethylene 30 propylene (FEP), perfluoroalkoxy (PFA), polyfluorovinylidene (PVDF), a modified copolymer of ethylene and tetrafluoroethylene (ETFE) or also ethylene/chlorotrifluoroethylene (ECTFE).

15. Device according to claim 1, further comprising a series of locations all containing the same reagent allowing a series of identical tests, a location containing a reference and a location intended to receive the waste products.

16. Device according to claim 1, further comprising a series of locations each containing different reagents each allowing a single type of test, one location containing a reference and one location intended to receive the waste products.

17. Process for biological analyses that implements a device as described in claim 1.

18. Automatic machine for analysis of biological liquid, that implements a device as described in claim 1.

19. Device according to claim 1, wherein the biological sample(s) is/are blood.

20. Device according to claim 1, wherein the biological sample(s) is/are whole blood.
